SEQUENCE LISTING (14 sequences)

SEQ ID NO: 1 - DNA, 1257 bp, Polaromonas sp. JS666
  source 1..1257: /organism=“Polaromonas sp. JS666” /mol_type=“unassigned DNA”
atgagtgaagcgattgtggtaaacaaccaaaacgaccaaagcagggcatacgcgatcccgcttgaggacattgatgtaagcaatccggagctgtttcgcgacaatacgatgtggggttattttgagcgtctgcgccgcgaagaccccgtgcattactgtaaggacagcttgtttggtccgtactggtcggtgaccaagttcaaggacatcatgcaggtggagacccatccggagatattttcatccgagggcaatatcaccatcatggagtccaatgcggcggtaaccctgccgatgttcattgcgatggatccgcccaagcacgacgtgcagcgcatggcggtcagtccgatcgtggcgccggagaacctcgccaagctcgaaggtctgatccgcgagcgtaccggtcgtgcgctggatggcctgccgatcaacgagacctttgactgggtcaagctcgtttcgatcaacctgacgacgcagatgctggcgacgctgtttgatttcccttgggaagaccgtgccaagctgacgcgctggtcggatgtcgcgacggcgctggtcggcacgggcattattgattcggaagagcagcgcatggaggagctcaaggggtgcgtgcaatacatgacccggctgtggaacgagcgcgtcaatgtgccaccgggcaatgatctgatatcgatgatggcgcacaccgagtccatgcgcaacatgacgccggaagagtttctgggcaacctcattttgctgatcgtcggcggcaatgacacgacccgcaactcgatgaccggcggcgtgctggcgctcaacgaaaatccggacgaataccgcaagctgtgcgccaacccggcgctgatcgcctccatggtgccggagatcgttcgttggcagacaccgctggcgcacatgcggcgtaccgcgctgcaggacaccgagctcggcggcaagtccattcgcaagggtgacaaggtcatcatgtggtatgtctccggcaaccgtgatcccgaagcgattgaaaatccggacgcgttcatcattgatcgcgccaagccgcgccatcacctctcgttcggtttcggcattcaccgctgcgtgggcaaccgtctcgccgagttgcagctgcgcatcgtttgggaggagttgctcaagcgctggcccaatccaggtcagatcgaggtcgttggcgcgcccgagcgcgtgctgtcgccctttgtgaagggctatgagtcgctgcccgtccgcatcaacgcttga

SEQ ID NO: 2 - DNA, 1218 bp, Polaromonas sp. JS666
  source 1..1218: /organism=“Polaromonas sp. JS666” /mol_type=“unassigned DNA”
gtgagcgaaactgtgattattgccggcgccggtcaggcggccggccaggcggttgcgagcctgcggcaagagggattcgacgggcgcatcgtgctggtcggcgccgagccggtgttgccgtatcagcgcccgccgctgtcgaaggcatttttggcgggcaccttgccgctggagcgattgttcctgaagccgccggcattctacgagcaggcgcgtgtggacacgctgctcggggtggccgtcaccgaacttgatgccgcccggcggcaggtgaggctggacgatggccgcgaactggcgtttgatcatctgctgctggcgactggcgggcgtgcccgtcggcttgactgcccgggtgccgaccatccgcgcctgcactatctgcgcaccgtggctgatgttgacggcattcgtgccgctctgcgtcccggggcccggctggtgctgatcggcggcggctacgtcggactcgagatcgccgccgtggccgccaaactggggctcgcggtgaccgtgctggaagcggcgccgacggtgctggcgcgtgtcacttgtccggccgtggcgcgcttcttcgaaagcgtgcaccggcaggcgggcgtgacgatccgctgcgcgacgacggtctccggcatcgagggcgatgcttcgctggcgcgggtcgtgaccggcgatggcgaacgcattgacgccgacctggtcattgccggcatcggtctgctgccgaacgtcgagttggcgcaggccgcgggtctggtctgcgacaacggcatcgtcgtcgacgaggaatgccggacctctgtgcccggcattttcgcggctggcgactgcacgcagcatccgaacgcgatctacgacagtcggctgcgtctcgaatcggtgcacaacgccattgagcagggcaagacggcggcggcggccatgtgtggcaaggccaggccgtatcggcaggtgccgtggttctggtccgatcagtacgacctcaagttacaaaccgcgggactcaaccgcggctatgaccaggtcgtgatgcggggcagtaccgacaaccgttcgtttgcggcgttctacctgcgcgacgggcgattgcttgccgtcgatgcggtcaaccgcccggtcgagttcatggtggccaaagcgctgattgcgaaccgcaccgtcatcgcgcccgagcggctcgccgacgagcgtatcgcagcgaaggacctggccggctga

SEQ ID NO: 3 - DNA, 321 bp, Polaromonas sp. JS666
  source 1..321: /organism=“Polaromonas sp. JS666” /mol_type=“unassigned DNA”
atgacaaaagttacttttattgaacacaatggtacggtccgcaacgtggacgtcgacgacggcctgtcggtgatggaggccgccgtcaacaacctggtgccgggcatcgatggcgactgcggtggcgcctgcgcctgcgccacctgccatgtgcacatcgacgccgcctggctggacaagttgccgccgatggaggcgatggaaaagtcgatgcttgagtttgccgagggccgcaacgaaagctcgcgcctgggttgtcagatcaagctcagccccgcgcttgacggcattgtggtgcgcacgccgctcggccagcactga

SEQ ID NO: 4 - DNA, 1209 bp, Pseudomonas putida
  source 1..1209: /organism=“Pseudomonas putida” /mol_type=“unassigned DNA”
gtgaacgcaaacgacagcggctgggaaggcaatatccggttggtgggggatgcgacggtaattccccatcacctaccaccgctatccaaagcttacttggccggcaaagccacagcggaaagcctgtacctgagaaccccagatgcctatgcagcgcagaacatccaactactcggaggcacacaggtaacggctatcaaccgcgaccgacagcaagtaatcctatcggatggccgggcactggattacgaccggctggtattggctaccggagggcgtccaagacccctaccggtggccagtggcgcagttggaaaggcgaacaactttcgatacctgcgcacactcgaggacgccgagtgcattcgccggcagctgattgcggataaccgtctggtggtgattggtggcggctacattggccttgaagtggctgccaccgccatcaaggcgaacatgcacgtcaccctgcttgatacggcagcccgggttctggagcgggttaccgccccgccggtatcggccttttacgagcacctacaccgcgaagccggcgttgacatacgaaccggcacgcaggtgtgcgggttcgagatgtcgaccgaccaacagaaggttactgccgtcctctgcgaggacggcacaaggctgccagcggatctggtaatcgccgggattggcctgataccaaactgcgagttggccagtgcggccggcctgcaggttgataacggcatcgtgatcaacgaacacatgcagacctctgatcccttgatcatggccgtcggcgactgtgcccgatttcacagtcagctctatgaccgctgggtgcgtatcgaatcggtgcccaatgccttggagcaggcacgaaagatcgccgccatcctctgtggcaaggtgccacgcgatgaggcggcgccctggttctggtccgatcagtatgagatcggattgaagatggtcggactgtccgaagggtacgaccggatcattgtccgcggctctttggcgcaacccgacttcagcgttttctacctgcagggagaccgggtattggcggtcgatacagtgaaccgtccagtggagttcaaccagtcaaaacaaataatcacggatcgtttgccggttgaaccaaacctactcggtgacgaaagcgtgccgttaaaggaaatcatcgccgccgccaaagctgaactgagtagtgcctga

SEQ ID NO: 5 - DNA, 324 bp, Pseudomonas putida
  source 1..324: /organism=“Pseudomonas putida” /mol_type=“unassigned DNA”
atgtctaaagtagtgtatgtgtcacatgatggaacgcgtcgcgaactggatgtggcggatggcgtcagcctgatgcaggctgcagtctccaatggtatctacgatattgtcggtgattgtggcggcagcgccagctgtgccacctgccatgtctatgtgaacgaagcgttcacggacaaggtgcccgccgccaacgagcgggaaatcggcatgctggagtgcgtcacggccgaactgaagccgaacagcaggctctgctgccagatcatcatgacgcccgagctggatggcatcgtggtcgatgttcccgataggcaatggtaa

SEQ ID NO: 6 - protein, 418 aa, Polaromonas sp. JS666
  [CDS]1..1257 from SEQ ID NO 1
MSEAIVVNNQNDQSRAYAIPLEDIDVSNPELFRDNTMWGYFERLRREDPVHYCKDSLFGPYWSVTKFKDIMQVETHPEIFSSEGNITIMESNAAVTLPMFIAMDPPKHDVQRMAVSPIVAPENLAKLEGLIRERTGRALDGLPINETFDWVKLVSINLTTQMLATLFDFPWEDRAKLTRWSDVATALVGTGIIDSEEQRMEELKGCVQYMTRLWNERVNVPPGNDLISMMAHTESMRNMTPEEFLGNLILLIVGGNDTTRNSMTGGVLALNENPDEYRKLCANPALIASMVPEIVRWQTPLAHMRRTALQDTELGGKSIRKGDKVIMWYVSGNRDPEAIENPDAFIIDRAKPRHHLSFGFGIHRCVGNRLAELQLRIVWEELLKRWPNPGQIEVVGAPERVLSPFVKGYESLPVRINA

SEQ ID NO: 7 - protein, 405 aa, Polaromonas sp. JS666
  [CDS]1..1218 from SEQ ID NO 2
VSETVIIAGAGQAAGQAVASLRQEGFDGRIVLVGAEPVLPYQRPPLSKAFLAGTLPLERLFLKPPAFYEQARVDTLLGVAVTELDAARRQVRLDDGRELAFDHLLLATGGRARRLDCPGADHPRLHYLRTVADVDGIRAALRPGARLVLIGGGYVGLEIAAVAAKLGLAVTVLEAAPTVLARVTCPAVARFFESVHRQAGVTIRCATTVSGIEGDASLARVVTGDGERIDADLVIAGIGLLPNVELAQAAGLVCDNGIVVDEECRTSVPGIFAAGDCTQHPNAIYDSRLRLESVHNAIEQGKTAAAAMCGKARPYRQVPWFWSDQYDLKLQTAGLNRGYDQVVMRGSTDNRSFAAFYLRDGRLLAVDAVNRPVEFMVAKALIANRTVIAPERLADERIAAKDLAG

SEQ ID NO: 8 - protein, 106 aa, Polaromonas sp. JS666
  [CDS]1..321 from SEQ ID NO 3
MTKVTFIEHNGTVRNVDVDDGLSVMEAAVNNLVPGIDGDCGGACACATCHVHIDAAWLDKLPPMEAMEKSMLEFAEGRNESSRLGCQIKLSPALDGIVVRTPLGQH

SEQ ID NO: 9 - protein, 107 aa, Pseudomonas putida
  [CDS]1..324 from SEQ ID NO 5
MSKVVYVSHDGTRRELDVADGVSLMQAAVSNGIYDIVGDCGGSASCATCHVYVNEAFTDKVPAANEREIGMLECVTAELKPNSRLCCQIIMTPELDGIVVDVPDRQW

SEQ ID NO: 10 - protein, 402 aa, Pseudomonas putida
  [CDS]1..1209 from SEQ ID NO 4
VNANDSGWEGNIRLVGDATVIPHHLPPLSKAYLAGKATAESLYLRTPDAYAAQNIQLLGGTQVTAINRDRQQVILSDGRALDYDRLVLATGGRPRPLPVASGAVGKANNFRYLRTLEDAECIRRQLIADNRLVVIGGGYIGLEVAATAIKANMHVTLLDTAARVLERVTAPPVSAFYEHLHREAGVDIRTGTQVCGFEMSTDQQKVTAVLCEDGTRLPADLVIAGIGLIPNCELASAAGLQVDNGIVINEHMQTSDPLIMAVGDCARFHSQLYDRWVRIESVPNALEQARKIAAILCGKVPRDEAAPWFWSDQYEIGLKMVGLSEGYDRIIVRGSLAQPDFSVFYLQGDRVLAVDTVNRPVEFNQSKQIITDRLPVEPNLLGDESVPLKEIIAAAKAELSSA

SEQ ID NO: 11 - DNA, 46 bp, Artificial Sequence
  source 1..46: /organism=“Artificial Sequence” /note=“PCR-Primer” /mol_type=“unassigned DNA”
ggtcatatgagatcattaatgagtgaagcgattgtggtaaacaacc

SEQ ID NO: 12 - DNA, 28 bp, Artificial Sequence
  source 1..28: /organism=“Artificial Sequence” /note=“PCR-Primer” /mol_type=“unassigned DNA”
agctaagctttcagtgctggccgagcgg

SEQ ID NO: 13 - DNA, 41 bp, Artificial Sequence
  source 1..41: /organism=“Artificial Sequence” /note=“PCR-Primer” /mol_type=“unassigned DNA”
ggtcatatgaccgaaatgacggtggccgccagcgacgcgac

SEQ ID NO: 14 - DNA, 34 bp, Artificial Sequence
  source 1..34: /organism=“Artificial Sequence” /note=“PCR-Primer” /mol_type=“unassigned DNA”
agctaagcttctaatgttgtgcagctggtgtccg

(19) United States
(12) Patent Application Publication
(10) Pub. No.: US 2016/0201093 A1
(43) Pub. Date: Jul. 14, 2016

(54) Process for the Bioconversion of C3-C13 Alkanes to C3-C13 Primary Alcohols

(71) Applicant: BASF SE, Ludwigshafen (DE)

(72) Inventors: Michael Breuer, Darmstadt (DE); Boris Breitscheidel, Waldsee (DE); Hans-Günter Wagner, Neuleiningen (DE); Detlef Kratz, Heidelberg (DE); Bernhard Hauer, Fußgönheim (DE); Daniel Scheps, Stuttgart (DE); Bernd Nebel, Stuttgart (DE)

(73) Assignee: BASF SE, Ludwigshafen (DE)

(21) Appl. No.: 14/908,293

(22) PCT Filed: Jul. 21, 2014
(86) PCT No.: PCT/EP2014/065580

§ 371 Date: Jan. 28, 2016

(30) Foreign Application Priority Data
Jul. 31, 2013 (EP) 13178725.1
Aug. 8, 2013 (EP) 13179721.9
Feb. 26, 2014 (EP) 14156695.0

(51) Int. Cl. C12P 7/16; C12P 7/04

(52) U.S. Cl. CPC C12P 7/16; C12P 7/04

(57) ABSTRACT

A process for preparing linear or branched primary alcohols with 3 to 13 carbon atoms from linear or branched alkanes with 3 to 13 carbon atoms by incubating a host organism having a functional P153 enzyme under elevated pressure in the presence of oxygen.

This application claims priority to European applications 13178725.1—filed on 31 Jul. 2013, 13179721.9—filed on 8 Aug. 2013 and 14156695.0—filed on 26 Feb. 2014, all of which are incorporated by reference in their entirety.

The present invention relates to a novel process for the bioconversion of linear or branched alkanes with 3 to 13 carbon atoms to linear or branched primary alcohols 5 with 3 to 13 carbon atoms.

### STATE OF THE ART

Linear or branched primary alcohols with 3 to 13 carbon atoms, e.g. 1-butanol, 2-ethylhexanol or 2-propylheptanol, are versatile chemical intermediates or raw materials for the production of plasticizers and solvent for paints, coating and varnishes. They also provide innovative products for a multitude of industrial applications, such as the manufacturing of plastics, textiles, cosmetics, drugs, antibiotics, vitamins, hormones, brake fluids and coatings

Linear or branched primary alcohols with 3 to 13 carbon atoms are generally produced by chemocatalysis. The most important chemical process for the production of linear or branched primary alcohols with 3 to 13 carbon atoms is the oxo-synthesis (hydroformylation) of linear or branched alkenes.

Over the last few years substantial progress has been made in the biotechnological production of bio-based linear or branched primary alcohols with 3 to 13 carbon atoms, e.g. 1-butanol, launching industrial initiatives like Gevo, Cobalt Technologies, Butyl Fuel LLC, Green Biologics, Syntec Biofuel, Tetravitae Bioscience, Butalco GmbH, METabolicEXplorer, Butamax Advance Biofules of BP and DuPont, to name just a few, which aim to commercialize bio-based linear or branched primary alcohols with 3 to 13 carbon atoms. At the same time new innovative attempts have been reported for the non-fermentative production of butanol in simpler organisms like Escherichia coli (E. coli). Although E. coli does not naturally produce butanol, it can be endowed by meta-bolic engineering or heterologous expression approaches either with genes coding for butanol formation activity or oxygenases like the cytochrome P450 monooxygenases (CYPs). In this light engineered E. coli strains comprising a set of genes involved in the biosynthesis of metabolic pathways have been described to produce 1.2 g butanol L^{-1[8,8]}. Another metabolic engineering-based approach for production of linear or branched primary alcohols with 3 to 13 carbon atoms, e.g. 1-butanol, makes use of the highly active amino acid biosynthetic pathway combining 2-ketoacid decarboxylases

with alcohol dehydrogenases for the transformation of common 2-keto acids I101. An alternative route was opened up by the functional reversal of the β-oxidation cycle in E co/that can be used as a metabolic platform for the synthesis of alcohols like 1-butanol and carboxylic acids with various chain lengths and functionalities.^{[11].}

Recently the ω-hydroxylations of medium chain alkanes by CYP153 enzymes from Mycobacterium marinum (CYP153A 16) and Polaromonas sp. was reported^{[15].}

### Objective

It is an objective of the present invention to provide an effective process for the production of linear or branched primary alcohols with 3 to 13 carbon atoms an a bio-based technology starting from economical resources.

### Subject Matter of the Invention

The object is achieved in accordance with the claims by a process for preparing linear or branched primary alcohols with 3 to 13 carbon atoms from linear or branched alkanes with 3 to 13 carbon atoms by incubating a host organism having a functional P153 enzyme under elevated pressure in the presence of oxygen.

The host organism can be a native or a recombinant microorganism. Bacteria are preferred as microorganisms. In case of native host organisms such microorganisms which have the ability to metabolize alkanes by a P153 enzyme system such as aerobic prokaryotes e.g. Pseudomonas and Mycobacteria are selected.

In case of a recombinant host organism a candidate is selected upon the industrial requirements such as simple cultivation conditions, fast growth rates and the availability of molecular genetic tools for strain manipulation. Especially preferred as a host organism is Escherichia coli.

The host organism must have a functional P153 enzyme.

Functional P153 enzyme means an enzyme of the CYP family, which are bacterial class I P450 monooxygenases that operate as three-component systems, comprised by the P450 itself and two additional redox proteins, namely an iron-sulfur electron carrier (ferredoxin) and a FAD-containing reductase (ferredoxin reductase) which are necessary for the transfer of electrons from NAD(P)H to the P450 active site^{[16].}

For a functional P153 enzyme one can use the P450 enzyme of one organism and the two redox proteins—ferredoxin and ferredoxin reductase—from the same organism. However, it is also possible to use the redox proteins from an organism different from the one of the P450 enzyme. For example the P450 enzyme of Polaromonas sp. can be functionally reconstituted with the redox proteins of Pseudomonas putida CamA and CamB^{[16].}

A functional P153 enzyme comprises three components irrespective of their original genetic source which allow an electron transfer from NAD(P)H to the P450 enzyme.

A preferred functional P153 enzyme is the one from Polaromonas sp (CYP153A P sp.)

SEQ ID NO:1 discloses the CYP153A gene of Polaromonas sp.

The ferredoxin and ferredoxin reductase genes of Polaromonas sp. are disclosed in SEQ ID NO:2 and NO:3 respectively.

The putidaredoxin reductase gene (CamA) of Pseudomonas putida is disclosed in SEQ ID NO:4

The putidaredoxin gene (CamB) of Pseudomonas putida is disclosed in SEQ ID NO:5.

Another preferred functional P153 enzyme is CYP153A6-BM01 which is disclosed in detail in^{[17].} a CYP153 enzyme carrying a point mutation (substitution A94V). The document^{[17]} is incorporated by reference herewith with respect to the cloning and expression of CYP153A6-BMO1.

The functionality of the P153 enzyme expressed in the host organism can be tested by CO difference spectral analyses. CO difference spectral analyses showed that cell extracts of CYP153A P sp. and CYP153A6-BM01 (0.2 9 cww ml⁻¹) expressed in E. coli BL21(DE3) yield soluble and active enzyme of 2.8 μM and 3.1 μM, respectively. This indicates that both cytochrome P450 monooxygenases were functionally expressed in similar yields. The monooxygenases were also stable. After a period of 24 hours at 30° C. we could determine more than 90% active biocatalyst. These results are not consistent with stability profiles of other members of the CYP153A subfamily, such as CYP153A16 from Mycobacterium marinum M [^{16, 25}] (fatty acid hydroxylase) and CYP153A from Acinetobacter sp. OC4 which possess less than 50% activity after 19 hours^{[19]}. The expression of the natural redox partners of each CYP153 enzyme was verified by SDS-PAGE (data not shown). Constant protein levels were determined.

According to the present invention linear or branched alkanes with 3 to 13 carbon atoms are used as starting materials for the production of the linear or branched primary alcohols with 3 to 13 carbon atoms. The alkane used as starting compound for the conversion into the respective alcohol should have the same carbon chain length and branching degree as the desired alcohol. So n-butane is used for the manufacture of n-butanol and n-heptane is used for the manufacture of n-heptanol and so forth. Preferred linear or branched alkanes with 3 to 13 carbon atoms according to the present invention are listed below.

Alkanes with 3 carbon atoms:

n-propane

Alkanes with 4 carbon atoms:

n-butane, 2-methylpropane (iso-butane)

Alkanes with 5 carbon atoms:

n-pentane, 2-methylbutane

Alkanes with 6 carbon atoms:

n-hexane, 2-methylpentane, 3-methylpentane, 2,2-dimethylbutane, 2,3-dimethylbutane

Alkanes with 7 carbon atoms:

n-heptane, 2-methylhexane, 3-methylhexane, 2,2-dimethylpentane, 2,3-dimethyl-pentane, 2,4-dimethylpentane, 3,3-dimethylpentane, 3-ethyl pentane. 2,2,3-trimethyl-butane

Alkanes with 8 carbon atoms:

n-octane, methylheptane, e.g. 2-methylheptane, dimethyhexanes, e.g. 2,2-dimethylhexane, ethylhexanes, e.g. 2-ethylhexane, trimethylpentanes, e.g. 2,2,3-trimethylpentane, methylethylpentanes, e.g. 2-methyl-3-ethyl-pentane

Alkanes with 9 carbon atoms:

n-nonane, methyloctanes, e.g. 2-methyloktane, dimethyiheptanes, e.g. 2,3-dimethylheptane, ethyiheptanes, e.g. 2-ethylheptane, methylethyihexanes, e.g. 2-methyl-3-ethylhexane, diethylpentanes, e.g. 3,3-diethylpentane

Alkanes with 10 carbon atoms:

n-decane, methylnonanes, e.g. 2-methylnonane, dimethyloktanes, e.g. 2,3-dimethyloktane, methylethylheptanes, e.g. 2-methyl-3-ethylheptane, propylhexanes, e.g. 2-propyihexane, isopropyl hexanes, e.g. 2-isopropylhexane, methylpropylpentanes, e.g. 2-propyl-4-methylpentane and 2-propyl-5-methylpentane

Alkanes with 11 carbon atoms:

n-undecane, iso-undecanes

Alkanes with 12 carbon atoms:

n-dodecane, iso-dodecanes

Alkanes with 13 carbon atoms:

n-tridecane, iso-tridecanes

According to the present invention the linear or branched alkanes with 3 to 13 carbon atoms can be used as starting material alone or as mixtures of two or more linear or branched alkanes with 3 to 13 carbon atoms in order to manufacture mixtures of two or more linear or branched alcohols.

More preferred n-butane, n-octane, 2-ethylhexane, n-nonane, n-decane, mixtures of n-nonane, methyloctanes, e.g. 2-methyloktane, dimethyiheptanes, e.g. 2,3-dimethylheptane, and ethylheptanes, e.g. 2-ethyiheptane, and mixtures of propylhexanes, e.g. 2-propylhexane, isopropyl hexanes, e.g. 2-isopropylhexane, methylpropylpentanes, e.g. 2-propyl-4-methylpentane and 2-propyl-5-methylpentane are used as linear or branched alkanes with 3 to 13 carbon atoms.

Most preferred n-butane, n-octane, 2-ethylhexane, n-nonane and n-decane are used as linear or branched alkanes with 3 to 13 carbon atoms.

The process according to the invention can be carried out at temperatures from 0 to 50° C., preferably from 5 to 40° C., and most preferred from 15 to 30° C.

The process according to the invention uses preferably resting host organism cells which were suspended in an aqueous buffer solution, preferably in potassium phosphate pH=7.5.

The process according to the invention introduces a hydroxyl group into a linear or branched alkane with 3 to 13 carbon atoms by an enzymatic oxidation. Therefore molecular oxygen has to be present in the reaction medium in order to provide the necessary oxygen atom for the hydroxyl group. The molecular oxygen is usually fed to the reaction system in form of synthetic air preferrably together with a stream of the linear or branched alkane with 3 to 13 carbon atoms. The alkane/air gas stream usually consists of 0, 1% to 50.0% alkane and 50.0% to 99.9% synthetic air, preferably 0.5% to 20.0% alkane and 80.0% to 99.5% synthetic air, more preferably 1.0% to 10.0% alkane and 90.0% to 99.0% synthetic air, and most preferably 1.0% to 3.0% alkane and 97.0% to 99.0% synthetic air. Particularly, the alkane/air gas stream consists of 2.0% alkane and 98.0% synthetic air. All percentage values are volume percent.

The inlet flow rate of the alkane/air gas stream usually amounts from 1 to 10.000 L gas×L⁻¹ reaction volume×h⁻¹, preferably from 5 to 5000 L gas×L⁻¹ reaction volume×h⁻¹, more preferably from 10 to 1000 L gas×L⁻¹ reaction volume×h⁻¹, and most preferably from 50 to 500 L gas×L⁻¹ reaction volume×h⁻¹. Particularly, the inlet flow rate of the alkane/air gas stream amounts from 100 to 300 L gas×L⁻¹ reaction volume×h⁻¹.

Alkanes, which are not gaseous at the reaction temperature, preferably are fed as liquids to the reaction system. In this case nitrogen is used as carrier gas together with synthetic air. The nitrogen/air gas stream usually consists of 0.1% to 50.0% nitrogen and 50.0% to 99.9% synthetic air, preferably 0.5% to 20.0% nitrogen and 80.0% to 99.5% synthetic air, more preferably 1.0% to 10.0% nitrogen and 90.0% to 99.0% synthetic air, and most preferably 1.0% to 3.0% nitrogen and 97.0% to 99.0% synthetic air. Particularly, the nitrogen/air gas stream consists of 2.0% nitrogen and 98.0% synthetic air. All percentage values are volume percent.

The solubility of alkanes in water or aqueous media is rather low and thus, constitutes a critical parameter for a biocatalytic process. In an attempt to enhance substrate availability, we performed additional in vivo experiments under pressure conditions using a high pressure reactor tank. Although it is well understood that high pressure conditions can denature enzymes we tested the applicability of elevated pressure in our process.

Elevated pressure shall mean that the overall pressure in the reaction system is above the atmospheric pressure. The overall pressure in the reaction system is caused by the alkane applied, by the oxygen needed for the hydroxylation reaction and by the nitrogen used when reacting alkanes which are nor gaseous at reaction temperature. Preferably a mixture of alkane and synthetic air is preformed and applied to the reaction system affecting a selected pressure between 1 and 25, preferably between 2 and 20 and most preferred between 3 and 15 bar.

The best product yields were obtained at a pressure of 15 bar, experiments carried out at more than 20 bar caused a decrease in production of linear or branched primary alcohols with 3 to 13 carbon atoms. The productivity in 100 mM KiP04 biotransformation medium remarkably increased product formation from 10.4 mM (120 mmol primary alcohol (g_{cww})-1 h-1) to 17.8 mM (210 mmol primary alcohol (g_{cww})-1 h-1). A maximum of 0.6 g primary alcohol L-1 after 24 h reaction time was obtained using the monooxygenase enzymes and a cell mass of 30 g_{cww} E. coli resting cells which was increased to 1.3 g L-1 at 15 bar pressure. This results represent a raise in yield by a factor of >2 and productivity of 0.15 g L-1 h-1 in a time course of 2-8 hours (linear increase of product was measurable) without further oxidation and reaction of the primary alcohol product. Remarkable to us was the fact that our enzymatic systems were feasible to oxidize small alkanes at low temperatures (0° C.) giving us insights into the system in the sense that the production in resting cell starts without a lack phase at the beginning where the temperature is still low, what confirm other in vitro oxidation results^{[38].} The small overall concentrations at higher pressure conditions might be explained by cell disruption caused by the additional shear stress and/or by a reduced transport of metabolic key intermediates like C02, which lead to a metabolic repression. Through continuous sampling and re-pressurizing with synthetic air we assured oxygen supply for the oxidation process. In contrast to the fermentation assembly under normal pressure, we were not able to remove the alcohol product during the process potentially leading to leakage of ions and the considerable disruption of cell metabolism caused by holes in bacterial membranes^{[39]}.

The process according to the invention oxidizes linear or branched alkanes with 3 to 13 carbon atoms preferably to linear or branched primary alcohols with 3 to 13 carbon atoms. Dependent of the reaction conditions minor amounts of linear or branched secondary alcohols with 3 to 13 carbon atoms (usually less than 15%, preferably less than 10% of the amount of linear or branched primary alcohols with 3 to 13 carbon atoms) can also be detected.

For some applications the mixtures of linear or branched primary alcohols with 3 to 13 carbon atoms and linear or branched secondary alcohols with 3 to 13 carbon atoms can be used without further purification. In case pure linear or branched primary alcohols with 3 to 13 carbon atoms are wanted the reaction mixture can be purified by techniques well known to the skilled person such as distillation.

According to the present invention linear or branched primary alcohols with 3 to 13 carbon atoms are obtained as reaction products. Preferred linear or branched primary alcohols with 3 to 13 carbon atoms obtained by the present invention are listed below.

Alcohols with 3 carbon atoms:

1-propanol

Alcohols with 4 carbon atoms:

1-butanol, 2-methyl-1-propanol (iso-butanol)

Alcohols with 5 carbon atoms:

pentanol, 2-methyl-1-butanol Alcohols with 6 carbon atoms:

hexanol, 2-methyl-1-pentanol, 3-methyl-1-pentanol, 2,2-dimethyl-1-butanol, 2,3-dimethyl-1-butanol

Alcohols with 7 carbon atoms:

1-heptanol, 2-methyl-1-hexanol, 3-methyl-1-hexanol, 2,2-dimethyl-1-pentanol, 2,3-dimethyl-1-pentanol, 2,4-dimethyl-1-pentanol, 3,3-dimethyl-1-pentanol, 3-ethyl-1-pentanol, 2,2,3-trimethyl-1-butanol

Alcohols with 8 carbon atoms:

octanol, methyl-1-heptanols, e.g. 2-methyl-1-heptanol, dimethyl-1-hexanols, e.g. 2,2-dimethyl-1-hexanol, ethyl-1-hexanols, e.g. 2-ethyl-1-hexanol, trimethyl-1-pentanols, e.g. 2,2,3-trimethyl-1-pentanol, methylethyl-1-pentanols, e.g. 2-methyl-3-ethyl-1-pentanol

Alcohols with 9 carbon atoms:

1-nonanol, methyl-1-octanols, e.g. 2-methyl-1-oktanol, dimethyl-1-heptanols, e.g. 2,3-dimethyl-1-heptanol, ethyl-1-heptanols, e.g. 2-ethyl-1-heptanol, methylethyl-1-hexanols, e.g. 2-methyl-3-ethyl-1-hexanol, diethyl-1-pentanols, e.g. 3,3-diethyl-1-pentanol Alcohols with 10 carbon atoms: 1-decanol, methyl-1-nonanols, e.g. 2-methyl-1-nonanol, dimethyl-1-oktanols, e.g. 2,3-dimethyl-1-oktanol, methylethyl-1-heptanols, e.g. 2-methyl-3-ethyl-1-heptanol, propyl-1-hexanols, e.g. 2-propyl-1-hexanol, isopropyl-1-hexanols, e.g. 2-isopropyl-1-hexanol, methylpropyl-1-pentanols, e.g. 2-propyl-4-methyl-1-propanol and 2-propyl-5-methyl-1-hexanol

Alcohols with 11 carbon atoms:

1-undecanol, iso-1-undecanols

Alcohols with 12 carbon atoms:

1-dodecanol, iso-1-dodecanols

Alcohols with 13 carbon atoms:

1-tridecanol, iso-1-tridecanols

More preferred 1-butanol, 1-octanol, 2-ethyl-1-hexanol, 1-nonanol, 1-decanol, mixtures of 1-nonanol, methyl-1-octanols, e.g. 2-methyl-1-oktanol, dimethyl-1-heptanols, e.g. 2,3-dimethyl-1-heptanol, and ethyl-1-heptanols, e.g. 2-ethyl-1-heptanol, and mixtures of propyl-1-hexanols, e.g. 2-propyl-1-hexanol, isopropyl-1-hexanol, e.g. 2-isopropyl-1-hexanol, methylpropyl-1-pentanols, e.g. 2-propyl-4-methyl-1-pentanol and 2-propyl-5-methyl-1-pentanol are used as linear or branched alkanes with 3 to 13 carbon atoms.

Most preferred 1-butanol, 1-octanol, 2-ethyl-1-hexanol, 1-nonanol and 1-decanol are used as linear or branched alkanes with 3 to 13 carbon atoms.

### WORKING EXAMPLES

### Example 1

### Cloning of CYP153A and CYP153A6

The enzyme CYP153A P. sp. (Bpro_5301) and the corresponding redox system with a FAO-dependent oxidoreductase (Bpro_530) and a ferredoxin (Bpro_299) from Polaromonas sp. strain JS666 ATCC BAA-500 were introduced into the Nda and Hina111 cloning sites of the pET-28a-(+) vector. The coding genes were amplified by PCR using oligonucleotides 5′-GGT CAT ATG AGA TCA TTA ATG AGT GAA GCG ATT GTG GTA AAC AAC C-3′ (SEQ 10 NO:11) and 5′-AGCT AAGCTTTCA GTGCTGGCCGAG CGG-3′ (SEQ 10 NO:12). The enzyme CYP153A6 (ahpG) and the natural redox system with a FAO-dependent oxidoreductase (ahpH) and a ferredoxin (ahpI) from Mycobactedum sp. HXN-1500 was also cloned with the Nda and Hina111 cloning sites of the pET-22b-(+) vector. The genes coding for the operon were amplified by PCR using oligonucleotides 5′-GGTCATATGACCGAAATGACGGTGGCCGCCAGCGAC-GCGAC-3′ (SEQ ID NO:13) and 5′-AGCT AAGCTTCTA ATG TTG TGC AGC TGG TGT CCG-3′ (SEQ ID NO:14). The following steps are similar to the one explained above. The ligated plasmids were used to transform competent E. coli OH5a cells via heat shock. Successful cloning was verified by automated ONA-sequencing (GATC-Biotech, Konstanz, Germany).

### Example 2

### Determination of P450

Concentrations of the P450 enzymes were determined by the carbon monoxide (CO) differential spectral assay, based on the formation of the characteristic Fe11-CO complex at 448 nm. The cells were disrupted by sonication on ice (4×2 min, 2 min intervals). Enzymes in cell-free extracts were reduced by the addition of 10 mM dithionite from a freshly prepared 1 M stock solution, and the carbon monoxide complex was formed by slow bubbling with CO gas for approximately 30 s. The concentrations were calculated using the absorbance difference at A₄₅₀ and A₄₉₀ (Ultrospec 3100pro spectrophotometer, Amersham Biosciences) and an extinction coefficient of 91 M⁻¹ cm-1^{[22].}

### Example 3

### Cultivation of CYP153A Cells

1 μl Plasmid was used to transform 10 μl competent E. coli BL21(DE 3) cells for the in vivo experiments. After 60 min regeneration in 90 μl SOC-media, 100 μL were used to start the 5 ml LB preculture, which was cultivated at 37° C. and 180 rpm.

One milliliter preculture was used to inoculate the main culture. Cultivations for whole cell bioconversions were carried out in 1 L Erlenmeyer shake flasks containing 200 ml TB and eM9Ymedia supplemented with the appropriate antibiotics. The growth was carried out on a shaker to an 00500 of 1.1-1.3. Expression was induced by the addition of 0.25 mM IPTG. The culture was supplemented with 4 g L-1 glycerol, 0.5 mM 5-aminolevulinic acid (o-ALA) and 100 mg FeSO4 in E. coli: The cells were incubated for 24 hours at 28° C. and 180 rpm and harvested by a centrifugation step at 4.000×g and 4° C. for 30 min.

Due to variations in the expression level of the different CYP153A variants, 2-3 independently cultured were prepared to assure a high enzyme concentration. The pellets were washed with 100 mM potassium phosphate buffer (pH 7.4) or eM9 media. After this procedure the cells were concentrated into 100 ml eM9 or 100 mM potassium phosphate buffer pH=7.5 to an end concentration of 30 g_{cww} L⁻¹ buffer or media. After the cells were provided with 1% glycerol (v/v) and 20 mM glucose carbon source, the gaseous substrate was added to the reaction mixture. Samples were taken after 1, 2, 4, 8 and 24 h reaction time.

### Example 4

### In Vivo Biotransformations of Butane to 1-Butanol in E. coli with CYP153A

For bioconversions of gaseous alkanes, 100 ml of cell suspension and 15 μl of antifoam 204 (Sigma-Aldrich) were stirred in a 250 ml Schott-flask at room temperature. Butane was added to the reaction mix with different inlet gas ratios of 1-10% butane and 90-99% synthetic air. The gas flow rate was also varied from 10-50 1×h⁻¹ (corresponds to 40 to 200 L gas×L⁻¹ reaction volume×h^{-I}) by using a Bronkhorst mass flow unit in order to elucidate the optimum conditions. Butane/air gas supply into the cell slurry was guaranteed through a continuous flow rate and the use of a sparger after mixing in a dispenser nozzle. To minimize product lass, a back flow cooling system was used. After defined time point's samples from the bioreactor flask or the wash flask, which was installed downstream of the fermentation flask to assure product removal, were taken and after a fast and tight sealing procedure analyzed by GC/MS-headspace chromatography.

Biotransformations were carried out with resting cells in 100 mM potassium phosphate buffer pH 7.5. We observed that the addition of a small amount of alkane. 1 mM hexane, for adaption of cells through the normal growth process and product formation is advantageous. For the quantification of the product the concentrations of 1-butanol and 2-butanol in the reaction and downstream flasks were combined. The total amount of butanol isomers formed during reaction is named “butanol all up” in the following text.

In order to examine the ability of an E. coli host system to produce 1-butanol with the heterologous expressed CYP153A enzymes, we performed a biotransformation for 24 h under continuous gas flow, atmospheric pressure and different culture media conditions. Butanol yields were enhanced by improving the fermentation assembly through the increase of the inlet gas flow rate and aeration as well as the implementation of product removal. Butane gas and air were supplied at rates of 10, 30, 40 or 50 L h⁻¹. The maximum product yield was observed at 50 1×h⁻¹ (corresponds to 200 L gas×L⁻¹ reaction volume×h⁻¹) and a butane-air ratio of 2:98.

Under these conditions it was possible to minimize oxygen-transfer limitations. The use of a sparger unit contributed to higher product formation rates owing to an increased aeration. The exposure of whole cells to 1-butanol over long time periods negatively influenced the total product yields obtained in our experiments. Without implementation of product removal, a total product concentration of 70% (7.8-8.2 mM, unpublished data) was accomplished. A fast and reliable product removal enables constant 1-butanol production by preventing cell damage and cell death due to an accumulation of polar products in the cell membrane^{[26, 27]}.

Also the addition of a glycerol/glucose mixture, reported to have a beneficial effect on cell function and nicotinamide cofactor regeneration, was investigated |²⁰1. Due to the fact that glycerol is known to be a driving force for cofactor regeneration in whole cellmediated redox biocatalysis |²⁸1, media containing either 0.05-0.3% glucose, 0.5-2% glycerol or a mixture of glucose/glycerol were tested. In the absence of glycerol or glucose butanol concentrations less than 0.5 mM were detected. A mixture of 20 mM glucose and 1% glycerol was determined to be the most efficient carbon source concentration for butanol production. Carbon source depletion was not observed studying 12 and 14 hour biotransformation experiments.

The transformation of butane to 1-butanol by CYP153A6-BM01 during the first 4 hours was more efficient in minimal-salt eM9 (10.7 mM butanol per 30 9 cww) than in 100 mM KP04 medium (7 mM butanol per 30 9 cww). To avoid amino acid catabolized repression experiments were not performed in the fermentation medium eM9Y containing yeast extract The experiments with CYP153A P. sp. results in 9 mM butanol per 30 9 cww with eM9 and 5.4 mM butanol per 30 Qcww in 100 mM potassium phosphate. CYP153A P. sp. showing a noticeable slower production rate (up to 25%) compared to CYP153A6-BM01. From the results obtained, we believe that the medium composition strengthens the cofactor regeneration system of the whole cell system. Resting cells for biotransformations in 100 mM potassium phosphate medium were grown prior in terrific broth medium comprising a rather complex and rich medium and thus might achieve positive overall effects.

Under the optimized conditions described above we detected that CYP153A6-BM01 produced a maximum of 12.1 mM 1-butanol (29 mg 1-butanol per 9 cww resting cells) after 8 hours in 100 mM potassium phosphate biotransformation medium. In comparison, the product yield in minimal-salt medium eM9 reached a maximum of 10.3 mM 1-butanol (25 mg 1-butanol per gcww resting cells) after 4 hours reaction time. Thereafter a strong decrease in productivity was detected over time. Experiments using CYP153A P sp. resulted in product yields of 9 mM 1-butanol in eM9 and 10.4 mM 1-butanol in 100 mM KP04. respectively, within 4 hours reaction time, equivalent to 19.3 mg and 22.2 mg 1-butanol per gcww resting cells. In comparison to CYP153A6-BM01, CYP153A P sp. displayed approximately 10% lower butane conversion with a ω-regioselectivity of 86% (90% ω-regioselectivity of CYP153A6-BM01)^{[17]}. By using CYP153A6-BM01 we obtained a yield of 0.9 g 1-butanol L⁻¹, being similar to the activity reported for an engineered P450-BM3 variant (15 mM with 4 gcdw L⁻¹ in 4 hours)^{[29].}

The latter enzyme is known to hydroxylate propane and higher alkanes primarily at the more energetically favorable subterminal positions (ω-1, ω-2, ω-3)^{[21,30]}, whereas enzymes of the CYP153A subfamily offer preferred ω-regioselectivities. In terms of productivity, conversions in eM9 medium resulted in concentrations of 495 mmol 1-butanol (gcww)-1 h-1 for CYP153A6-BM01 and 315 mmol for CYP153A P sp., respectively. In contrast, 119 mmol 1-butanol (gcww)-1 h-1 were obtained with the best engineered P450BM3 variant under similar media conditions [29]. Another attractive feature of these hydroxylation reactions is that they are very selective and products do not suffer from overoxidation. No oxidation to butanal or butanoic acid and further reaction to 1,4-butanediol was detected. However, we cannot exclude the formation of such by products after having monitored the presence of these in in vitro experiments (might be utilized by the whole cells as carbon or energy sources^{[16]}.

### Example 5

### In Vivo Biotransformations of Butane to 1-Butanol Under Pressure

The hydroxylation of the gaseous substrate butane was also performed in a high pressure reactor. The cells were expressed as previously described mixed in 100 mM potassium phosphate buffer pH 7.5. 10 g of liquid butane in excess was added as a second phase at a temperature of −5° C. In a following step the pressure tanks (Carl Roth, high-pressure auto-clave 11) were sealed with the stainless steel caps connected via high pressure lines to a synthetic air gas cylinder, which makes it possible to apply a selected pressure between 1 bar to the reaction mixture. This step ensures also the supply of sufficient oxygen for the reaction. The (de)compression process at the beginning and during every sampling step was made as slowly as possible.

Analytics

To avoid product loss due to evaporation upon sampling and typical organic solvent extraction, we have established a GC/MS headspace method for product analysis. Samples were analyzed on a GC/MS QP-2010 instrument (Shimadzu, Japan) equipped with a FS-Supreme-5-column (30 m×0.25 mm×0.25 μm, Chromatographie Service GmbH, Langerwehe, Germany) and with a CombiPal Sampler operated in headspace mode and with a 2.5 ml tight gas syringe. Electron impact (EI) ionization and helium as carrier gas (flow rate 0.69 ml/min) were used. Mass units were monitored from 20 to 200 mlz and ionized at 70 eV. The injector and detector temperatures were set at 250° C. with a split-ratio of 15:1. One millilitre of the fermentation culture was transferred into a 20 ml headspace vial. After the addition of 100 μl of the internal standard (10 mM hexanol), the vials were capped. Temperature program: 40° C., hold 5 min, 5° C./min to 85° C., hold 1 min, 60° C./min to 300° C. For quantification of the small volatile compounds, the detector response was calibrated with the internal standard hexanol. A series of standard solutions with varied concentrations (0.01-2 mM of 1-butanol and 2-butanol) in 100 mM potassium phosphate buffer or in eM9 media were generated and analyzed by GC/MS. The stock solutions were kept always between 4° C. and were stable for at least 1 week.

Glucose and glycerol concentrations in the aqueous phase were determined by HPLC using 5 mM sulfuric acid as mobile phase. Cells from the fermentation fractions were separated from the supernatant by centrifugation at 20.000×g for 1 minute (Centrifuge 5417 C, Eppendorf, Germany). The supernatant was transferred into a new plastic tube, mixed with the internal standard xylitol to a final concentration of 10 mM and finally sterile filtered. HPLC analysis was carried out on an Agilent System (1200 series) using the cation exchange resin column Aminex HPX-87H (300×7.8 mm, Bio-Rad, USA) at 60° C. and a flow rate of 0.5 ml/min. The substrates and products were quantified using the corresponding standards and a refractive index detector (Agilent 1200series, G1262A).

TABLE 1
In vivo butane oxidation yields of CYP153A P sp. with different pressure
conditions

| | CYP153A | P. sp. | |
|---|---|---|---|
| Pressure | Biotransformation media | 1-butanol [mM] | |
| atmospheric pressure | KiPO₄ | 10.4 ± 1.0 | (11) |
| 5 bar | KiPO₄ | 13.8 | (10) |
| 10 bar | KiPO₄ | 15.9 ± 2.7 | (9) |
| 15 bar | KiPO₄ | 17.8 ± 2.1 | (9) |
| 20 bar | KiPO₄ | 12.73 ± 1.3 | (9) |

Total 1-butanol production in resting E. coli BL21 (DE cells with CYP153A P sp. Cells were resuspended in 100 mM potassium phosphate buffer with glucose/glycerol as carbon source after cultivation in TB. Different pressure conditions were investigated. Values in parentheses are the percentage of 2-butanol formed during hydroxylations. Only 1- and 2-butanol were analysed in detectable amounts.

### LITERATURE

[1]E. M. Green, Curr Opin Biotechnol 2011, 22, 337.
[2]R. Cascone, Chem Eng Prog 2008, S4.
[3]R. K. Saxena, P. Anand, S. Saran, J. Isar, Biotechnol Adv 2009, 27, 895.
[4]K. Weissermel, H.-J. u. Arpe, za105758a2d, Industrial organic chemistry, Wiley-VCH, Weinheim, 2003.
[5]T. Lutke-Eversloh, H. Bahl, Curr Opin Biotechnol 2011.
[6]P. Durre, Ann N YAcad Sci 2008, 1125, 353.
[7]E. T. Papoutsakis, Curr Opin Biotechnol 2008, 19, 420.
[8]S. Atsumi, A. F. Cann, M. R. Connor, C. R. Shen, K. M. Smith, M. P. Brynildsen, K. J. Chou, T. Hanai, J. C. Liao, Metab Eng 2008, 10, 305.
[9]S. Atsumi, T. Hanai, J. C. Liao, Nature 2008, 451, 86.
[10]C. R. Shen, J. C. Liao, Metab Eng 2008, 10, 312.
[11]C. Dellomonaco, J. M. Clomburg, E. N. Miller, R. Gonzalez, Nature 2011, 476, 355.
[12]J. B. van Beilen, E. G. Funhoff, Appl Microbiol Biotechnol 2007, 74, 13.
[13]N. Hamamura, R. T. Storfa, L. Semprini, D. J. Arp, Appi Environ Microbiol 1999, 65, 4586
[14]J. B. van Beilen, E. G. Funhoff, Curr Opin Biotechnol 2005, 16, 308.
[15]E. G. Funhoff, J. Salzmann, U. Bauer, B. Witholt, J. B. van Beilen, Enzyme and Microbial Technology 2007, 40, 806.
[16]D. Scheps, S. H. Malca, H. Hoffmann, B. M. Nestl, B. Hauer, Org Biomol Chem 2011, 9, 6727
[17]D. J. Koch, M. M. Chen, J. B. van Beilen, F. H. Arnold, Applied and Environmental Microbiology 2009, 75, 337
[18]M. Bordeaux, A. Galameau, F. Fajula, J. Drone, Angew Chem Int Ed Eng 2011, 50, 2075
[19]T. Fujii, T. Narikawa, F. Sumisa, A. Arisawa, K. Takeda, J. Kato, Biosci Biotechnol Biochem 2006, 70, 1379
[20]R. K. Gudiminchi, C. Randall, D. J. Opperman, O. A. Olaofe, S. T. Harrison, J. Albertyn, M. S. Smit, Appl Microbiol Biotechnol 2012
[21]P. Meinhold, M. W. Peters, A. Hartwick, A. R. Hernandez. F. H. Arnold, Advanced Synthesis & Catalysis 2006, 348, 763.
[22]T. Omura, R. Sato, J Biol Chem 1964, 239, 2379.
[23]N. Fujita, F. Sumisa, K. Shindo, H. Kabumoto, A. Arisawa, H. Ikenaga, N. Misawa, Biosci Biotechnol Biochem 2009, 73, 1825.
[24]D. Sirim, F. Wagner, A. Lisitsa, J. Pleiss, BMC Biochem 2009, 10, 27.
[25]S. Honda Malca, D. Scheps, L. Kuhnel, E. Venegas-Venegas, A. Seifert, B. M Nest!, B. Hauer, Chem Commun (Camb) 2012.
[26]S. Isken, J. A. de Bont, Extremophiles 1998, 2, 229.
[27]E. J. Steen, R. Chan, N. Prasad, S. Myers, C. J. Petzold, A. Redding, M. Ouellet, J D. Keasling, Microb Cell Fact 2008, 7, 36
[28]L. M. Blank, B. E. Ebert, B. Buhler, A. Schmid, Biotechnol Bioeng 2008, 100, 1050.
[29]R. Fasan, M. M. Chen, N. C. Crook, F. H. Arnold, Angew Chem Int Ed Engi 2007, 46, 8414.
[30] Fasan, Y. T. Meharenna, C. D. Snow, T. L. Poulos, F. H. Arnold, J Mol Biol 2008, 383, 1069.
[31] Kadkhodayan, E. D. Coulter, D. M. Maryniak, T. A. Bryson, J. H. Dawson, J Biol Chem 1995, 270, 28042.
[32]J. R.-O.-C. Michael J. Eisenmenger, Enzyme and Microbial Technology 2009, 45, 331.
[33]M. Summit, B. Scott, K. Nielson, E. Mathur. J. Barass, Extremoph!les 1998. 2, 339.
[34]J. Pelloux, C. Rusterucci. E. J. Mellerowicz, Trends Plant Sci 2007, 12, 267.
[35]Y. K. Cho, D. B. Northrop, Biochemistry 1999, 38, 10908.
[36]S. Dallet, M. D. Legoy, Biochim Biophys Acta 1996, 1294, 15.
[37]F. E. Zilly, J. P. Acevedo, W. Augustyniak, A. Deege, U. W. Hausig, M. T. Reetz, Angew Chem Int Ed Engl 2011, 50, 2720.
[38]S. Staudt, C. A. Muller, J. Marienhagen, C. Boing, S. Buchholz, U. Schwaneberg, H. Groger, Beilstein J Org Chem 2012, 8, 186.
[39] Grant, J. M. Woodley. F. Baganz, Enzyme Microb Technol 2011, 48, 480.

1. A process for preparing a linear or branched primary alcohol with 3 to 13 carbon atoms from a linear or branched alkane with 3 to 13 carbon atoms by incubating a host organism having a functional P153 enzyme under elevated pressure in the presence of oxygen.

2. The process according to claim 1, wherein the host organism is unable to use linear or branched primary alcohols with 3 to 13 carbon atoms as a carbon source.

3. The process according to claim 2, wherein the host organism is E. coli.

4. The process according to claim 1, wherein the pressure is from 2-20 bar.

5. The process according to claim 1, wherein the incubation temperature is from 0-50° C.

6. The process according to claim 1, wherein the P153 enzyme is isolated from the organism selected from the group of Pseudomonas, Polaromonas and Mycobacterium.

7. The process according to claim 6, wherein the P153 enzyme has a polypeptide sequence selected from the group which is formed by SEQ ID NO: 1, SEQ ID NO:2 and derivatives of SEQ ID NOS: 1 and 2 wherein the derivatives have up to three amino acid exchanges compared to SEQ ID NOS: 1 and 2.

8. The process according to claim 1, wherein a minor amount of a linear or branched secondary alcohol with 3 to 13 carbon atoms is produced in addition to the linear or branched primary alcohol with 3 to 13 carbon atoms.

9. The process according to claim 1, wherein the alkane is n-butane, n-octane, 2-ethylhexane, n-nonane, n-decane, a mixture of n-nonane, methyloctane, a dimethylheptane, and ethylheptane, or a mixture of propylhexane, isopropylhexane, and methylpropylpentane, and the linear or branched primary alcohol with 3 to 13 carbon atoms is 1-butanol, 1-octanol, 2-ethyl-1-hexanol, 1-nonanol, 1-decanol, a mixture of 1-nonanol, methyl-1-octanol, dimethyl-1-heptanol, and ethyl-1-heptanol, or a mixture of propyl-1-hexanol, isopropyl-1-hexanol, and methylpropyl-1-pentanol.

10. The process according to claim 1, wherein the alkane is n-butane, n-octane, 2-ethylhexane, n-nonane or n-decane, and the linear or branched primary alcohol is 1-butanol, 1-octanol, 2-ethyl-1-hexanol, 1-nonanol or 1-decanol.
